(19) United States
(12) Patent Application Publication
(10) Pub. No.: US 2021/0153570 A1
(43) Pub. Date: May 27, 2021

(54) THERMAL TREATMENT UNDERGARMENT

(71) Applicant: Brigid Delena Soldavini, Naples, FL (US)

(72) Inventor: Brigid Delena Soldavini, Naples, FL (US)

(21) Appl. No.: 16/947,220

(22) Filed: Jul. 23, 2020

Related U.S. Application Data
(60) Provisional application No. 62/939,002, filed on Nov. 22, 2019.

(51) Int. Cl. A41B 9/00; A41D 13/005

(52) U.S. Cl. CPC A41B 9/001; A41D 2400/10; A41B 2300/20; A41D 13/0058

(57) ABSTRACT

A thermal treatment undergarment retains a thermal treatment pack in position over a locus of pain or injury. The thermal treatment undergarment includes a first elastic layer, such as a brief, that may be worn. A thermal pouch is defined on the undergarment, such as by a joining seam of a second elastic layer, which may also be formed as a brief. A joining seam around the leg openings and the lateral sides provides an abdominal pocket and a back pocket. Likewise, a joining seam about the leg openings and each of an anterior and posterior portion of the brief, defines a lateral side pocket, for treatment of the hip pain or injury.

### CROSS-REFERENCE TO RELATED APPLICATION

This application claims the benefit of priority of U.S. provisional application No. 62/939,002 filed Nov. 22, 2019, the contents of which are herein incorporated by reference.

### BACKGROUND OF THE INVENTION

The present invention relates to undergarments, and more particularly to undergarments for pain relief.

For many application of thermal treatments, whether heat packs or ice packs, to alleviate lower abdominal pain can be difficult. The position on the body makes it difficult to retain the thermal packs to the locus of the pain.

For example, many women experience pain from menstrual cramps on a recurring basis. Likewise, women undergoing a Caesarian Section delivery may experience pain following their delivery. Other conditions, such as sciatica, hip replacement surgery and the like present the same challenge for both men and women.

Most thermal pack applications to the lower abdominal area restrict the individual's ability to remain mobile in caring for themselves or others, without removing the thermal pack.

While elastic bandages are typically used to position thermal packs, the thermal pack may still slip out or reposition itself during patient movements.

As can be seen, there is a need for an improved method and apparatus for placing and retaining a thermal treatment pack to an individual's body.

### SUMMARY OF THE INVENTION

In one aspect of the present invention, a thermal treatment undergarment is disclosed. The thermal treatment undergarment includes an outer layer formed of a fabric material having a pair of leg openings and a waist opening. An inner layer is dimensioned to substantially conform to the outer layer. The inner layer is joined to the outer layer about the pair of leg openings. A joining seam vertically extends from a point proximal to at least one of the leg openings and terminating proximal to the waist opening. The joining seam defines at least one pouch between the outer layer and the inner layer, the at least one pouch dimensioned to receive a thermal treatment pack.

In some embodiments, a thermal treatment pack is received within the at least one pouch. The thermal treatment pack may be one of an ice pack and a heat pack.

In some embodiments, the joining seam includes an anterior joining seam positioned forwardly of a lateral centerline of the thermal treatment under garment. A posterior joining seam is positioned aft of the lateral centerline of the thermal treatment under garment.

In some embodiments, the thermal treatment undergarment is formed as a brief. In other embodiments, the thermal treatment undergarment is formed as a compression short. In yet other embodiments, the thermal treatment undergarment is formed as a high waist panty.

In other embodiments, an elastic waistband is attached to the outer layer around the waist opening.

In other embodiments, the at least one pouch includes a plurality of pouches disposed in a spaced apart relation about a circumferential aspect of the thermal treatment under garment.

In other embodiments, the inner layer of the thermal treatment under garment is joined to the outer layer by a stitch extending around the pair of leg openings.

In a preferred embodiment, at least the outer layer is formed of an elastic material.

These and other features, aspects and advantages of the present invention will become better understood with reference to the following drawings, description and claims.

### BRIEF DESCRIPTION OF THE DRAWINGS

FIG. 1 is a Front Perspective View of the thermal treatment undergarment;

FIG. 2 is a Front View of the thermal treatment undergarment;

FIG. 3 is a Perspective View of the thermal treatment undergarment;

FIG. 4 is a Side Cross Section View of the thermal treatment undergarment; with an Ice Packs Being Inserted; and

FIG. 5 is an Alternate Embodiment of the thermal treatment undergarment; as Boxer Briefs.

### DETAILED DESCRIPTION

The following detailed description is of the best currently contemplated modes of carrying out exemplary embodiments of the invention. The description is not to be taken in a limiting sense, but is made merely for the purpose of illustrating the general principles of the invention.

Broadly, embodiments of the present invention provides a system, method, and apparatus for securing a thermal treatment pack to a user's lower abdominal area.

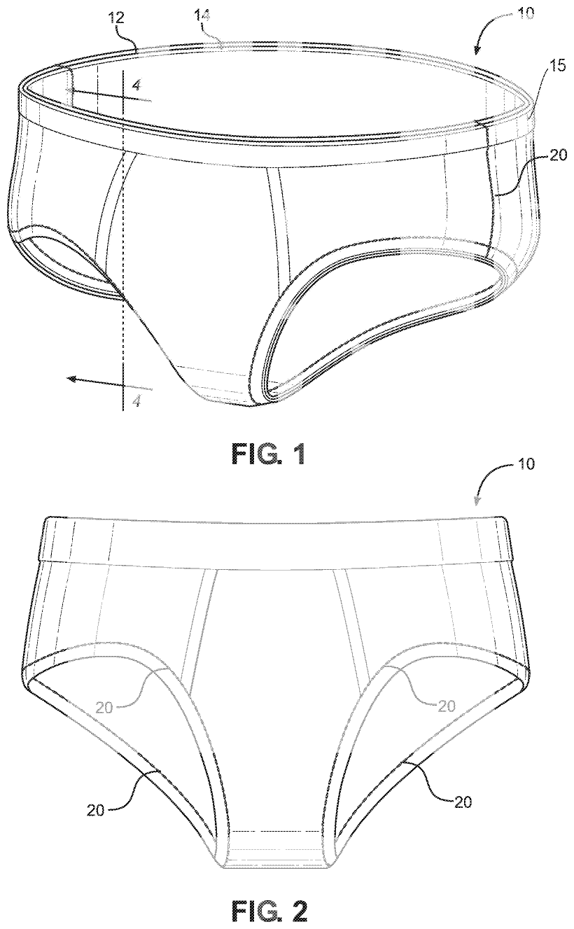

As seen in the non-limiting embodiment shown in reference to FIGS. 1 and 2, a thermal treatment undergarment 10 according to aspects of the present invention is shown. The thermal treatment undergarment 10 includes a brief that is formed of an elastic material. The brief 10 is provided with at least one retaining pouch 16 that is dimensioned to receive a thermal treatment pack 18. The thermal treatment pack 18 may be either a heat pack or a cold/ice pack. The retaining pouch 16 is positioned so that the thermal 18 pouch may be positioned over the locus of an abdominal injury or pain, such as cramps and the like.

In the non-limiting embodiment shown, thermal treatment undergarment 10 may be formed of an outer layer 12 having a pair of leg opening and a waist opening. A waistband 15 may be applied around the waist opening. An inner layer 14, substantially conforming to the outer layer 12, is joined to the outer layer 12 about the leg openings, essentially forming a two-layer brief. The outer layer 12 may then be sewn or joined to the inner layer 14, to form the one or more retaining pouches 16. In the embodiment shown, the outer layer 12 and the inner layer 14 are joined at the lateral sides, around the leg openings, and across the top of the crotch portion.

The waistband 15 of the first brief and an optional waistband 15′ of the second brief may be left open to provide a passage through which the thermal pack 18 is inserted into the one or more pouches 16. The thermal pack 18 may then be positioned over the locus of the pain source. The elastic tension of at least the outer layer 12 of the thermal treatment garment 10 will retain the thermal pack 18 in the selected position.

As will be appreciated, the location of the pouch 16 may be varied by selecting a vertically oriented joining seam 20 placement to correspond to the locus of the injury or pain. In the case of a hip injury, a lateral thermal treatment pouch may be formed by changing the placement of the lateral side joining seam 20, which is moved towards each of an anterior portion of the abdomen and towards a posterior portion of the buttocks. The leg openings may be left joined to define a pocket between the leg opening and the anterior/posterior joining seams 20. The lateral thermal treatment pouch 16 may then receive the thermal pack 18 and is retained in position over the hip area.

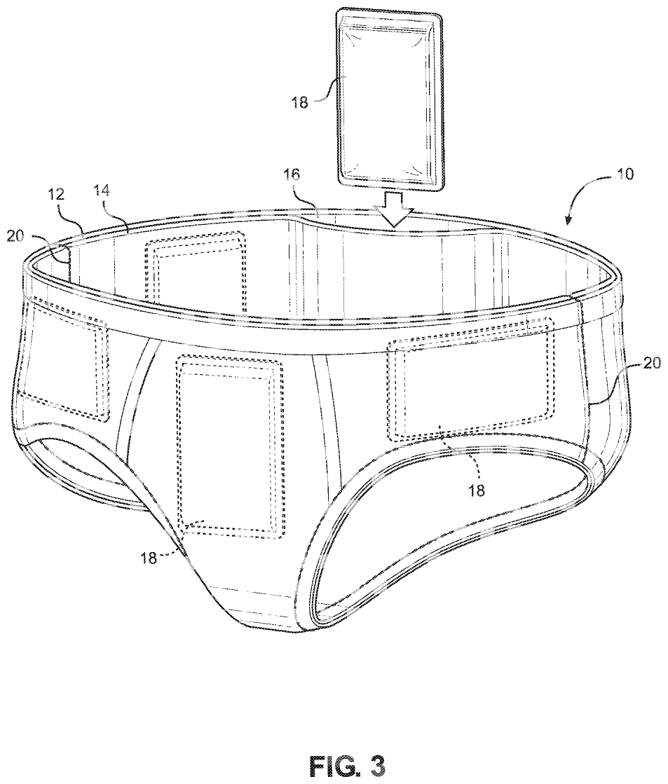

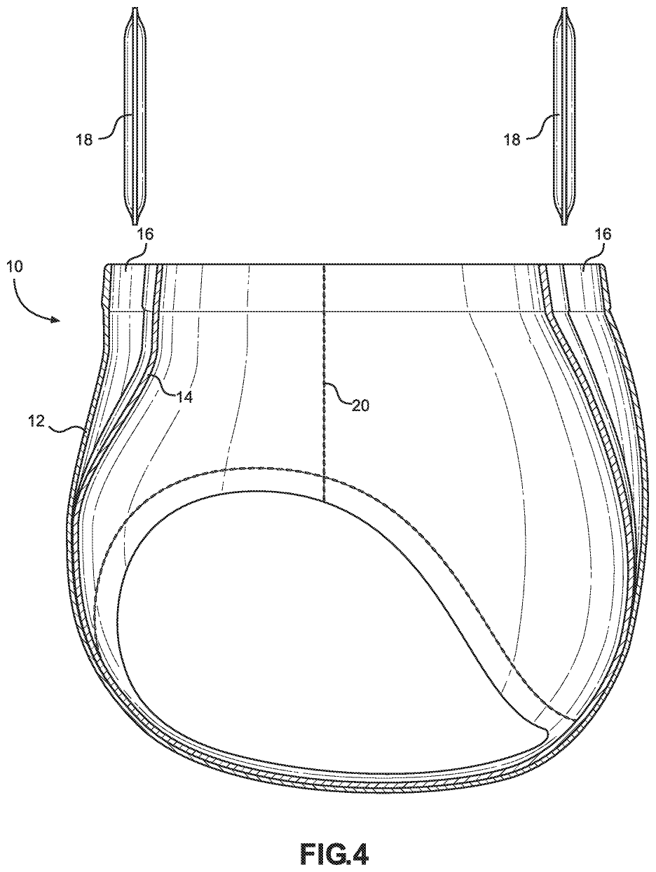

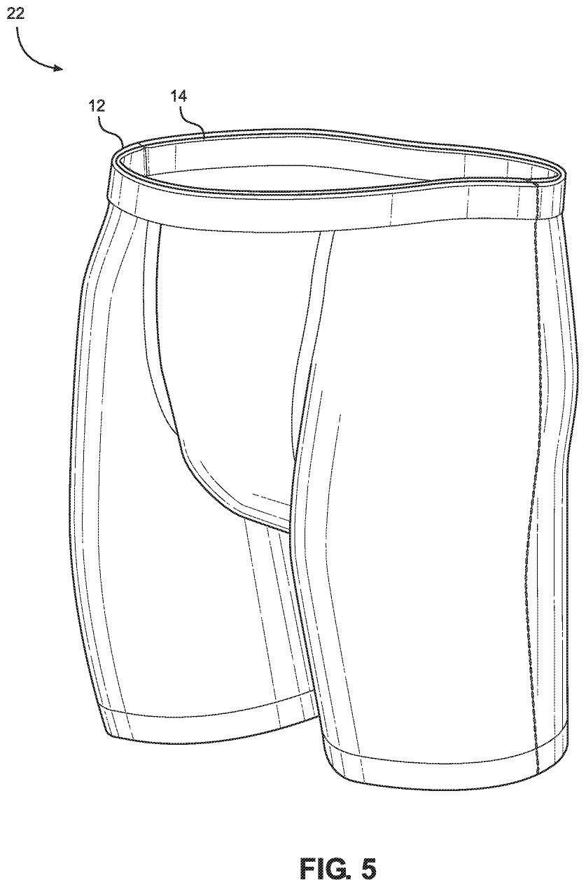

While shown as a brief in FIGS. 1-3, embodiments of the thermal treatment undergarment 10 may also include a high waist panty, such as shown in reference to FIG. 4, or a long or intermediate leg length compression short undergarment, such as shown in reference to FIG. 5. Regardless of the form factor for the thermal treatment undergarment 10, the outer layer 12 and the inner layer 14 may be joined to define the one or more pouches 16 at the desired locus of the injury or pain.

It should be understood, of course, that the foregoing relates to exemplary embodiments of the invention and that modifications may be made without departing from the spirit and scope of the invention.

What is claimed is:

1. A thermal treatment undergarment, comprising:
an outer layer of a fabric material having a pair of leg openings and a waist opening;
an inner layer dimensioned to substantially conform to the outer layer, the inner layer joined to the outer layer about the pair of leg openings; and
a joining seam vertically extending from a point proximal to at least one of the pair of leg openings and terminating proximal to the waist opening, the joining seam defining at least one pouch between the outer layer and the inner layer, the at least one pouch dimensioned to receive a thermal treatment pack.

2. The thermal treatment undergarment of claim 1, wherein the thermal treatment pack is received within the at least one pouch.

3. The thermal treatment undergarment of claim 2, wherein the thermal treatment pack is one of an ice pack and a heat pack.

4. The thermal treatment undergarment of claim 1, the joining seam further comprising:
an anterior joining seam positioned forwardly of a lateral centerline of the thermal treatment undergarment; and
a posterior joining seam positioned aft of the lateral centerline of the thermal treatment undergarment.

5. The thermal treatment undergarment of claim 4, wherein the thermal treatment undergarment is formed as a brief.

6. The thermal treatment undergarment of claim 4, wherein the thermal treatment undergarment is formed as a compression short.

7. The thermal treatment undergarment of claim 4, wherein the thermal treatment undergarment is formed as a high waist panty.

8. The thermal treatment undergarment of claim 4, further comprising:
an elastic waistband attached to the outer layer around the waist opening.

9. The thermal treatment undergarment of claim 8, wherein the at least one pouch comprises a plurality of pouches disposed in a spaced apart relation about a circumferential aspect of the thermal treatment undergarment.

10. The thermal treatment undergarment of claim 4, wherein the inner layer is joined to the outer layer by a stitch extending around the pair of leg openings.

11. The thermal treatment undergarment of claim 1, wherein at least the outer layer is formed of an elastic material.
